(19) United States
(12) Patent Application Publication
(10) Pub. No.: US 2026/0158116 A1
(43) Pub. Date: Jun. 11, 2026

(54) RECOMBINANT COLLAGEN IONIC LIQUID COMPOSITION, PRODUCTION METHOD THEREOF, AND USE THEREOF

(71) Applicant: THE ACTIONS COMPANY, Chengdu (CN)

(72) Inventors: Bing XUE, Chengdu (CN); Zhaosen ZHANG, Chengdu (CN); Kate Jie Zhang LI, Chengdu (CN); Steven Yan CHENG, Chengdu (CN)

(73) Assignee: THE ACTIONS COMPANY, Chengdu (CN)

(21) Appl. No.: 19/409,868

(22) Filed: Dec. 5, 2025

(30) Foreign Application Priority Data
Dec. 9, 2024 (CN) 202411794366.0

(51) Int. Cl. A61K 38/39; A61K 47/10; A61K 47/18; A61P 17/14

(52) U.S. Cl. CPC A61K 38/39; A61K 47/10; A61K 47/183; A61P 17/14

(57) ABSTRACT

The present disclosure provides a recombinant collagen ionic liquid composition, a preparation method thereof, and a use thereof. The present disclosure is related to the technical field of cosmetics. The recombinant collagen ionic liquid composition provided by the present disclosure, in parts by mass, comprises: 19.8 to 79.99 parts of a lysine citrate ionic liquid, 20 to 80 parts of glycerol, and 0.01 to 0.2 parts of a recombinant collagen. The lysine citrate ionic liquid consists essentially of lysine and citric acid, in a molar ratio of the lysine to the citric acid of (4 to 1):(2 to 1). The recombinant collagen ionic liquid composition provided by the present disclosure has good protein solubility, high transdermal efficiency, low skin irritation, a short production cycle, a simple process, and is suitable for continuous production.

### FIELD

The present disclosure relates to the technical field of cosmetics, in particular to a recombinant collagen ionic liquid composition, a production method thereof, and use thereof.

### BACKGROUND

Hair loss is a phenomenon in which hair sheds from the scalp. Physiological hair loss is the normal shedding of hair caused by human metabolism; and pathological hair loss refers to abnormal or excessive hair shedding caused by various factors such as genetics, hormonal changes, medical conditions, and nutritional deficiencies, leading to hair thinning and even baldness. Hair loss not only affects an individual's self-image but may also have profound psychological and social impacts. Drug treatment is a common method for the improvement and treatment of hair loss, owing to its advantages such as convenience, ease of use, and lasting effects. Among them, drugs such as minoxidil and finasteride have been proven to be effective in improving hair loss, but they have issues such as long treatment periods, adverse reactions (e.g., rash, headache, hirsutism, lower limb edema, and increased blood pressure), and usage restrictions (e.g., finasteride is not suitable for female). Therefore, there is a need to develop functional ingredients with high safety and few side effects for promoting hair growth, to address the shortcomings of existing methods.

Studies have shown that hair loss is mainly caused by the aging of hair follicle stem cells. Type 17 collagen (type XVII collagen), which has high biocompatibility and good efficacy, is a key molecule in preventing the aging of hair follicle stem cells (Science, 2016, 351, 6273), and is capable of maintaining the stemness characteristics of hair follicles and promoting hair regeneration. Recombinant collagen can be produced on a large scale with high purity and can effectively avoid the risk of pathogens. Therefore, recombinant collagen has broad application prospects in fields such as cosmetics and biomedicine. However, recombinant collagen has a large relative molecular mass and a complex structure, making it difficult to cross multiple biological barriers in the body, and it is easily metabolized and broken down by gastric acid, enzymes, and enterohepatic circulation. When used for promoting hair growth, focus should be placed on the effective delivery of type 17 collagen to the target skin area to maximize its effects. Microneedle is a common method for improving the transdermal delivery efficiency of macromolecular active ingredients such as proteins and polypeptides. However, it is highly destructive to the skin, may cause local tissue damage and pain, and also poses risks of cross-infection and allergic reactions. Ionic liquids have advantages such as strong dissolving capacity and good transdermal absorption-promoting properties. When combined with an active ingredient, they can be directly applied to the skin surface to deliver the active ingredient, thereby increasing targeting ability and reducing adverse reactions, which provides a new approach for the efficient delivery of collagen and the promotion of hair growth. However, current ionic liquid formulations may have strong irritating and sensitizing properties, resulting in poor applicability to the skin. Therefore, there is an urgent need for a system with a high transdermal delivery rate and low skin irritation that can ensure the efficient transdermal delivery of collagen while reducing allergic reactions and discomfort for the subject.

In view of this, it is of great significance to provide a method for producing a recombinant collagen ionic liquid composition having good protein solubility, high transdermal efficiency and low skin irritation, which has a short production cycle, a simple process, and is suitable for continuous production.

### SUMMARY

A first purpose of the present disclosure is to provide a recombinant collagen ionic liquid composition to solve the aforementioned technical problems.

A second purpose of the present disclosure is to provide a method for producing the aforementioned recombinant collagen ionic liquid composition.

A third purpose of the present disclosure is to provide use of the aforementioned recombinant collagen ionic liquid composition in promoting hair follicle repair and hair growth.

To achieve the above purposes, the present disclosure provides the following technical solutions:
In a first aspect, the present disclosure provides a recombinant collagen ionic liquid composition, comprising, in parts by mass: 19.8 to 79.99 parts of a lysine citrate ionic liquid, 20 to 80 parts of glycerol, and 0.01 to 0.2 parts of a recombinant collagen;
wherein the lysine citrate ionic liquid consists essentially of lysine and citric acid, in a molar ratio of the lysine to the citric acid of (4 to 1):(2 to 1).

In some further embodiments, the composition comprises, in parts by mass: 29.85 to 49.95 parts of the lysine citrate ionic liquid, 50 to 70 parts of the glycerol, and 0.05 to 0.15 parts of the recombinant collagen.

In some further embodiments, the lysine citrate ionic liquid is produced by a method comprising: mixing lysine, citric acid and water, subjecting the mixture to a reaction, and removing water after the reaction being completed to obtain the lysine citrate ionic liquid.

In some further embodiments, the reaction is performed at a temperature of 25° C. to 100° C., and
the reaction is performed for 12 to 24 h.

In some further embodiments, the removing water is performed by distillation under reduced pressure.

In some further embodiments, the recombinant collagen includes a type XVII recombinant collagen.

In some further embodiments, the composition further comprises, in parts by mass, 0 to parts of water.

In a second aspect, the present disclosure provides a method for producing the aforementioned recombinant collagen ionic liquid composition, comprising: mixing all components in formulated amounts to obtain the recombinant collagen ionic liquid composition.

In some further embodiments, components other than the recombinant collagen are mixed first, and the recombinant collagen is then added and mixed.

In a third aspect, the present disclosure provides use of the aforementioned recombinant collagen ionic liquid composition for promoting hair follicle repair and hair growth.

Compared to the prior art, the present disclosure has the following beneficial effects:

The recombinant collagen ionic liquid composition provided by the present disclosure consists essentially of a lysine citrate ionic liquid, glycerol, and a recombinant collagen in a specific proportion. Among them, the lysine citrate ionic liquid is a product of purely natural ingredients, has good biocompatibility and safety, and has advantages such as good skin permeability, non-irritation, and convenient administration. The composition can effectively reduce allergic and irritant reactions in subjects; it contains an active ingredient for promoting hair growth, is convenient to use, can be directly applied to the scalp area for administration, and has strong targeting ability and high hair-growth efficiency.

The method for producing the recombinant collagen ionic liquid composition provided by the present disclosure has a short production cycle, a simple process, is suitable for continuous production, and has a low cost.

### BRIEF DESCRIPTION OF DRAWINGS

To illustrate the technical solutions in the embodiments of the present disclosure or in the prior art more clearly, a brief introduction to the accompanying drawings required for describing the embodiments or the prior art is provided below. It should be apparent that the drawings in the following description are only some embodiments of the present disclosure. For a person of ordinary skill in the art, other drawings can also be obtained based on these drawings without making any inventive effort.

FIG. 1 is a hydrogen nuclear magnetic resonance spectrum of the lysine citrate ionic liquid;

FIG. 2 is a hydrogen nuclear magnetic resonance spectrum of the arginine caprylate ionic liquid;

FIG. 3 is a hydrogen nuclear magnetic resonance spectrum of the proline citrate ionic liquid;

FIG. 4 shows the lysine citrate ionic liquid;

FIG. 5 shows the recombinant collagen ionic liquid compositions;

FIG. 6 shows the result of the hair growth experiment on mice, wherein FIG. 6A to FIG. 6I show, after 12 days of administration, the back skin of the mice in the blank control group, the collagen group, the Examples 7-11 groups and the Comparative Example 4 and 5 groups, respectively; and FIG. 6J to FIG. 6R show, after 14 days of administration, the back skin of the mice in the blank control group, the collagen group, the Examples 7-11 groups and the Comparative Example 4 and 5 groups, respectively;

FIG. 7 shows a comparison of a subject before (FIG. 7A) and after (FIG. 7B) using the recombinant collagen ionic liquid composition in Test Example 3; and

FIG. 8 shows a comparison of a subject before (FIG. 8A) and after (FIG. 8B and FIG. 8C) using the recombinant collagen ionic liquid composition in Test Example 4.

### DETAILED DESCRIPTION

The embodiments of the present disclosure will be described in detail below in conjunction with embodiments and examples. However, a person skilled in the art will understand that the following embodiments and examples are for illustrative purposes only and should not be construed as limiting the scope of the present disclosure. All other embodiments obtained by a person of ordinary skill in the art based on the examples in the present disclosure without making inventive efforts fall within the protection scope of the present disclosure. Where conditions are not specified, the procedures are carried out under conventional conditions or conditions recommended by the manufacturer. Reagents or instruments for which the manufacturer is not specified are conventional products that can be purchased from the market.

In a first aspect, the present disclosure provides a recombinant collagen ionic liquid composition, consisting essentially of a lysine citrate ionic liquid, glycerol, and a recombinant collagen. In parts by mass, the parts by mass of the lysine citrate ionic liquid can be, but not limited to, for example, 19.8 parts, 40 parts, 60 parts, or 79.99 parts.

In the recombinant collagen ionic liquid composition, glycerol is preferably used as a solubilizer to increase the dissolution rate of collagen, optimize the color and luster of the composition, and reduce the viscosity of the composition. The parts by mass of glycerol can be, but not limited to, for example, 20 parts, 40 parts, 60 parts, or 80 parts.

The parts by mass of the recombinant collagen can be, but not limited to, for example, 0.01 parts, 0.05 parts, 0.1 parts, 0.15 parts, or 0.2 parts.

The lysine citrate ionic liquid consists essentially of lysine and citric acid, and a molar ratio of the lysine to the citric acid can be, but not limited to, for example, 4:1, 3:2, 2:3, or 1:2.

The recombinant collagen ionic liquid composition provided by the present disclosure can effectively reduce allergic and irritant reactions in subjects, promotes absorption of the recombinant collagen by scalp, and has good effects of promoting hair follicle growth and hair growth. In some optional embodiments, the composition comprises, in parts by mass, 29.85 to 49.95 parts of the lysine citrate ionic liquid, 50 to 70 parts of glycerol, and 0.05 to 0.15 parts of the recombinant collagen.

In some optional embodiments, a method for producing the lysine citrate ionic liquid comprises: mixing lysine, citric acid and water, subjecting the mixture to a reaction, and removing water after the reaction being completed to obtain the lysine citrate ionic liquid.

The method for producing the lysine citrate ionic liquid is simple, convenient, and has a low cost.

In some optional embodiments, the reaction can be performed at a temperature of, for example, 25° C., 50° C., 75° C., or 100° C.

The reaction can be performed for a duration of, for example, 12 h, 18 h, or 24 h.

In some optional embodiments, the removing water is performed by a method including but not limited to distillation under reduced pressure, or other methods for removing water known to those skilled in the art.

In some optional embodiments, the recombinant collagen includes a type XVII recombinant collagen.

In some optional embodiments, the composition further comprises, in parts by mass, 0 to 30 parts of water, for example, 0 part, 5 parts, 10 parts, 20 parts, 25 parts, or 30 parts.

In a second aspect, the present disclosure provides a method for producing the aforementioned recombinant collagen ionic liquid composition, comprising: mixing all components in formulated amounts to obtain the recombinant collagen ionic liquid composition.

The production method has a short cycle, a simple process, is suitable for continuous production, and has a low cost.

To ensure uniformity of the mixing of raw materials and the solubility and stability of the collagen in the composition, in some optional embodiments, components other than the recombinant collagen are mixed first, and the recombinant collagen is then added and mixed.

In a third aspect, the present disclosure provides use of the aforementioned recombinant collagen ionic liquid composition in promoting hair follicle repair and hair growth.

The recombinant collagen ionic liquid composition provided by the present disclosure can effectively reduce allergic and irritant reactions in subjects, promotes absorption of the recombinant collagen by scalp, and has good effects of promoting hair follicle growth and hair growth.

The present disclosure is further illustrated below by specific examples and comparative examples; however, it should be understood that these examples are for more detailed illustration only and should not be construed as limiting the present disclosure in any way.

### Example 1

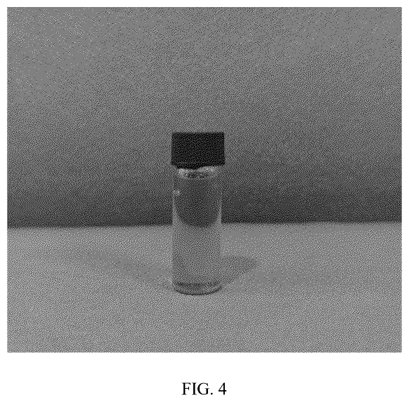

This example provided an amino acid ionic liquid, with an appearance as shown in FIG. 4, which was prepared mainly through the following steps:

Under a nitrogen atmosphere, 0.4 mol of lysine was added to a round-bottom flask, and 50 mL of deionized water was then added. The mixture was stirred and mixed thoroughly and evenly at a speed of 800 rpm.

0.1 mol of citric acid was dissolved in 50 mL of deionized water, and the resulting solution was added dropwise into the round-bottom flask and mixed uniformly. The mixture was then left to react at 80° C. for 12 h under magnetic stirring.

After the reaction was completed, most of the water was removed by rotary evaporation at 70° C., and residual water was then removed by drying in a vacuum oven at 60° C. for 48 h to obtain a lysine citrate ionic liquid (4:1).

### Example 2

This example provided an amino acid ionic liquid, which was prepared mainly through the following steps:

Under a nitrogen atmosphere, 0.3 mol of lysine was added to a round-bottom flask, and 50 mL of deionized water was then added. The mixture was stirred and mixed thoroughly and evenly at a speed of 800 rpm.

0.1 mol of citric acid was dissolved in 50 mL of deionized water, and the resulting solution was added dropwise into the round-bottom flask and mixed uniformly. The mixture was then left to react at 80° C. for 12 h under magnetic stirring.

After the reaction was completed, most of the water was removed by rotary evaporation at 70° C., and residual water was then removed by drying in a vacuum oven at 60° C. for 48 h to obtain a lysine citrate ionic liquid (3:1).

### Example 3

This example provided an amino acid ionic liquid, which was prepared mainly through the following steps:

Under a nitrogen atmosphere, 0.2 mol of lysine was added to a round-bottom flask, and 50 mL of deionized water was then added. The mixture was stirred and mixed thoroughly and evenly at a speed of 800 rpm.

0.1 mol of citric acid was dissolved in 50 mL of deionized water, and the resulting solution was added dropwise into the round-bottom flask and mixed uniformly. The mixture was then left to react at 80° C. for 12 h under magnetic stirring.

After the reaction was completed, most of the water was removed by rotary evaporation at 70° C., and residual water was then removed by drying in a vacuum oven at 60° C. for 48 h to obtain a lysine citrate ionic liquid (2:1).

### Example 4

This example provided an amino acid ionic liquid, which was prepared mainly through the following steps:

Under a nitrogen atmosphere, 0.1 mol of lysine was added to a round-bottom flask, and 50 mL of deionized water was then added. The mixture was stirred and mixed thoroughly and evenly at a speed of 800 rpm.

0.1 mol of citric acid was dissolved in 50 mL of deionized water, and the resulting solution was added dropwise into the round-bottom flask and mixed uniformly. The mixture was then left to react at 80° C. for 12 h under magnetic stirring.

After the reaction was completed, most of the water was removed by rotary evaporation at 70° C., and residual water was then removed by drying in a vacuum oven at 60° C. for 48 h to obtain a lysine citrate ionic liquid (1:1).

### Example 5

This example provided an amino acid ionic liquid, which was prepared mainly through the following steps:

Under a nitrogen atmosphere, 0.1 mol of lysine was added to a round-bottom flask, and 50 mL of deionized water was then added. The mixture was stirred and mixed thoroughly and evenly at a speed of 800 rpm.

0.15 mol of citric acid was dissolved in 50 mL of deionized water, and the resulting solution was added dropwise into the round-bottom flask and mixed uniformly. The mixture was then left to react at 25° C. for 24 h under magnetic stirring.

After the reaction was completed, most of the water was removed by rotary evaporation at 70° C., and residual water was then removed by drying in a vacuum oven at 60° C. for 48 h to obtain a lysine citrate ionic liquid (1:1.5).

### Example 6

This example provided an amino acid ionic liquid, which was prepared mainly through the following steps:
Under a nitrogen atmosphere, 0.1 mol of lysine was added to a round-bottom flask, and 50 mL of deionized water was then added. The mixture was stirred and mixed thoroughly and evenly at a speed of 800 rpm.
0.2 mol of citric acid was dissolved in 50 mL of deionized water, and the resulting solution was added dropwise into the round-bottom flask and mixed uniformly. The mixture was then left to react at 100° C. for 18 h under magnetic stirring.
After the reaction was completed, most of the water was removed by rotary evaporation at 70° C., and residual water was then removed by drying in a vacuum oven at 60° C. for 48 h to obtain a lysine citrate ionic liquid (1:2).

The amino acid ionic liquids prepared above were experimentally verified. The results showed that the ionic liquids were successfully prepared in all of Examples 1 to 6. Among them, the experimental results of Example 3 are as follows:

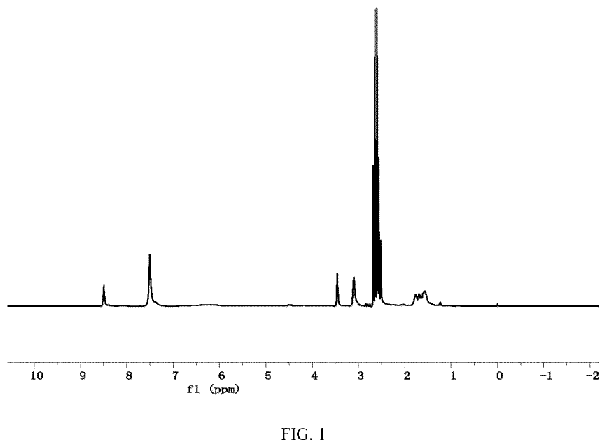

The hydrogen nuclear magnetic resonance (1H NMR) spectrum is shown in FIG. 1. The ¹H NMR spectrum of the lysine citrate ionic liquid shows signals corresponding to methylene at 1.95 to 1.43 ppm (m, —CH₂), 3.029 ppm (m, —CH₂), and 3.756 ppm (m, —CH₂). A signal corresponding to an amino group was detected at 7.46 ppm (s, —NH₂), representing a fragment of L-lysine. Signals corresponding to methylene at 2.757 ppm (q, —CH₂) and 2.662 ppm (q, —CH₂) were signals of citric acid-related fragments. A chemical shift of the carboxyl group of citric acid at 8.51 ppm (s, —COOH) indicated the successful synthesis of [L-Lys][CA]. These results indicated that the [L-Lys][CA] ionic liquid was successfully prepared.

### Comparative Example 1

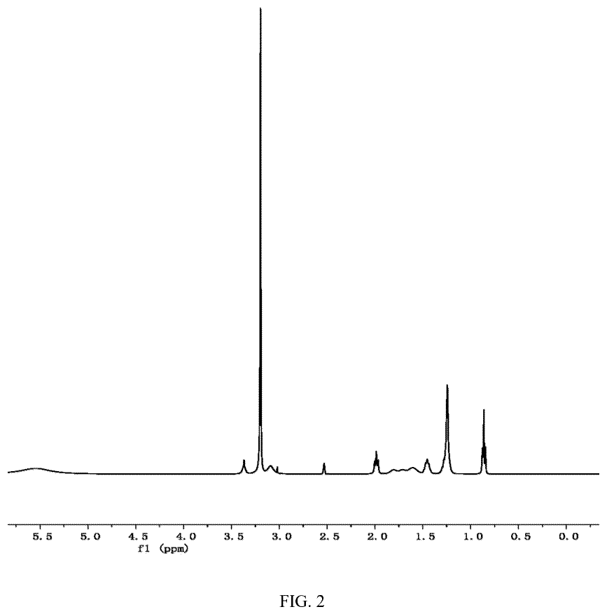

An arginine caprylate ionic liquid was prepared (the hydrogen nuclear magnetic resonance spectrum is shown in FIG. 2), which only differed from Example 2 in that lysine was replaced with arginine, and citric acid was replaced with caprylic acid.

### Comparative Example 2

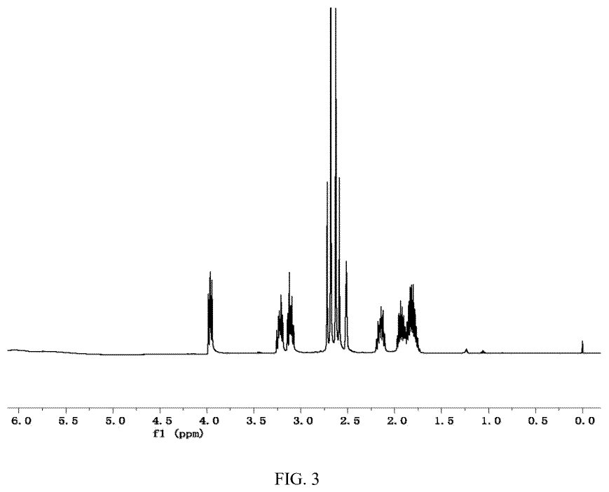

A proline citrate ionic liquid was prepared (the hydrogen nuclear magnetic resonance spectrum is shown in FIG. 3), which only differed from Example 2 in that lysine was replaced with proline.

### Comparative Example 3

An amino acid-based ionic liquid was prepared, which only differed Example 2 in that citric acid was replaced with butyric acid.

### Example 7

A recombinant collagen ionic liquid composition was prepared mainly through the following steps:

Water, glycerol, and the amino acid ionic liquid prepared in Example 1 were mixed uniformly to obtain a mixed solution. Then, a type XVII recombinant collagen was dissolved in the mixed solution and stirred at 30° C. for 10 h to obtain the recombinant collagen ionic liquid composition.

The mass ratio of water, glycerol, the amino acid ionic liquid, and the type XVII recombinant collagen was 10:70:20:0.1.

### Example 8

A recombinant collagen ionic liquid composition was prepared mainly through the following steps:

Water, glycerol, and the amino acid ionic liquid prepared in Example 2 were mixed uniformly to obtain a mixed solution. Then, a type XVII recombinant collagen was dissolved in the mixed solution and stirred at 30° C. for 10 h to obtain the recombinant collagen ionic liquid composition.

The mass ratio of water, glycerol, the amino acid ionic liquid, and the type XVII recombinant collagen was 5:60:35:0.1.

### Example 9

A recombinant collagen ionic liquid composition was prepared mainly through the following steps:

Water, glycerol, and the amino acid ionic liquid prepared in Example 3 were mixed uniformly to obtain a mixed solution. Then, a type XVII recombinant collagen was dissolved in the mixed solution and stirred at 30° C. for 10 h to obtain the recombinant collagen ionic liquid composition.

The mass ratio of water, glycerol, the amino acid ionic liquid, and the type XVII recombinant collagen was 30:50:19.8:0.2.

### Example 10

A recombinant collagen ionic liquid composition was prepared mainly through the following steps:

Glycerol and the amino acid ionic liquid prepared in Example 2 were mixed uniformly to obtain a mixed solution. Then, a type XVII recombinant collagen was dissolved in the mixed solution and stirred at 30° C. for 10 h to obtain the recombinant collagen ionic liquid composition.

The mass ratio of glycerol, the amino acid ionic liquid, and the type XVII recombinant collagen was 20:79.99:0.01.

### Example 11

A recombinant collagen ionic liquid composition was prepared mainly through the following steps:

Glycerol and the amino acid ionic liquid prepared in Example 3 were mixed uniformly to obtain a mixed solution. Then, a type XVII recombinant collagen was dissolved in the mixed solution and stirred at 30° C. for 10 h to obtain the recombinant collagen ionic liquid composition.

The mass ratio of glycerol, the amino acid ionic liquid, and the type XVII recombinant collagen was 80:19.9:0.1.

### Comparative Example 4

This comparative example differed from Example 8 in that the amino acid ionic liquid of Comparative Example 1 was used.

### Comparative Example 5

This comparative example differed from Example 8 in that the amino acid ionic liquid of Comparative Example 2 was used.

### Comparative Example 6

This comparative example differed from Example 8 in that the amino acid ionic liquid of Comparative Example 3 was used.

### Comparative Example 7

This comparative example differed from Example 8 in that a different amino acid ionic liquid was used. The amino acid ionic liquid was prepared as follows:

Under a nitrogen atmosphere, 0.1 mol of lysine was added to a round-bottom flask, and 50 mL of deionized water was then added. The mixture was stirred and mixed thoroughly and evenly at a speed of 800 rpm.

0.4 mol of citric acid was dissolved in 50 mL of deionized water, and the resulting solution was then added dropwise into the round-bottom flask and mixed uniformly. The mixture was then left to react at 100° C. for 18 hours under magnetic stirring.

After the reaction was completed, most of the water was removed by rotary evaporation at 70° C., and residual water was then removed by drying in a vacuum oven at 60° C. for 48 h to obtain a lysine citrate ionic liquid (1:4).

### Test Example 1

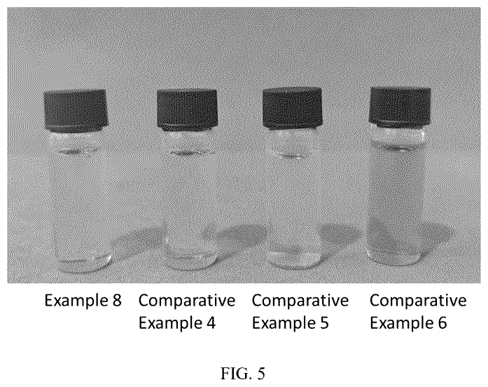

The effect of the recombinant collagen ionic liquid composition prepared in the present disclosure on promoting mouse hair growth was evaluated. Female C57BL/6J mice (6 to 8 weeks old, 18 to 20 g) were used. The hair on the back of the mice was shortened with an electric clipper, and a proper amount of depilatory cream was applied evenly to the clipped area to remove residual hair. The area was washed clean with warm water and wiped dry. The application area was about 3 cm×2 cm. The back skin of the mice was considered clean when it was smooth without breakage or residual hair. Subsequently, the depilated area and its surroundings were disinfected with 75% ethanol. Mice with pink skin and hair in the telogen phase were selected to establish a mouse model of hair loss. The next day, the mice were randomly divided into the following groups, with 3 mice in each group: a blank control group (administered with normal saline), a collagen group (administered with the recombinant collagen with a concentration consistent with Example 8), Example 7 to 11 groups, and Comparative Example 4 to 5 groups. The appearance of the products of Example 8 and Comparative Examples 4, 5, and 6 is shown in FIG. 5. In the depilated area on the back of the mice, the above products were applied for each group respectively, once a day, with an amount of 0.5 mL covering the depilated area, for 14 consecutive days. The changes in mouse hair were observed from day 1 to day 21.

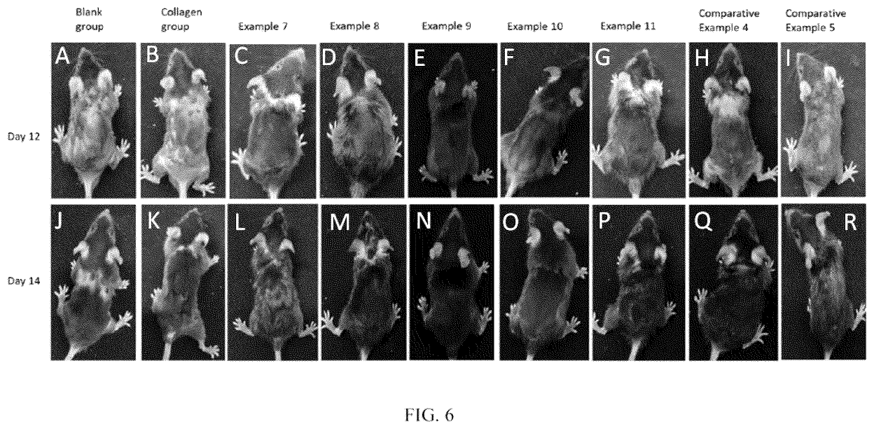

After 7 days of administration, the backs of the mice in the blank control group were still pink, with hair follicles in the telogen phase. The backs of the mice in the collagen group and the Example 10 group (with the lowest recombinant collagen content in the composition) showed a small amount of light gray color, indicating that collagen could promote the transition of hair from the telogen phase to the anagen phase, with a small number of hair follicles growing. The Example 7, 8, 9, and 11 groups and the Comparative Example 4 to 5 groups showed varying degrees of hair follicle growth. No significant difference was observed between the Example 7, 8, and 11 groups and the Comparative Example 4 and 5 groups. The light gray area on the backs of the mice in the Example 9 group was larger and more uniform, mainly due to the higher recombinant collagen content in the composition. As shown in FIG. 6, after 12 days of administration, the back skin of the mice in the blank control group, the collagen group, and the Example 10 group showed a light gray to gray color, with short hair growing in some areas. Among them, the collagen group and the Example 10 group were superior to the blank control group. The Example 7, 8, 9, and 11 groups and the Comparative Example 4 and 5 groups showed a better hair growth status, with the skin appearing dark gray to black. Among them, the hair growth in the Example 9 group was optimal. The hair growth in the Example 7, 8, and 11 groups showed no significant difference but were all superior to that in the Comparative Example 4 and groups. The hair growth in the Comparative Example 4 and 5 groups showed no significant difference but were both superior to that in the blank control group and the collagen group. After 14 days of administration, the hair of the mice in the blank control group and the collagen group was relatively short and did not fully recover to the pre-depilation level. The hair of some mice in the Example 10 group returned to a normal level. The hair of all mice in the Example 7, 8, 9, and 11 groups and the Comparative Example 4 and 5 groups returned to a normal level, appearing black-gray. After the administration was stopped, the mice were continuously observed. It was found that some mice in Comparative Example 5 began to lose hair, with sparse hair on their backs, while the mice in the Example 7 to 11 groups and the Comparative Example 4 group showed no hair loss. The above results indicate that ionic liquid compositions can promote hair growth in mice, and the ingredients and proportions of the compositions affect the effect and persistence of hair growth. The ionic liquid compositions of the present disclosure (Example 7 to 11 groups) have a better effect of promoting hair growth.

### Test Example 2

The effect of the recombinant collagen ionic liquid composition prepared in the present disclosure on promoting hair growth was evaluated. Eighty volunteers with hair loss problems, 25 to 55 years old, were recruited and divided into 4 groups, with 20 people in each group. The compositions of Comparative Example 4 and Examples 8, 9, and 11 were used respectively for 14 days or more (the pH of Comparative Examples 5 and 7 could not meet the requirement for skin care products (pH≥4.5) and could not be used in human trials; the odor of Comparative Example 6 was strong, causing reactions such as nausea, for which the subjects were unwilling to receive the product of Comparative Example 6). The composition was applied once a day, evenly covering the test area, and left on for 15 to 30 min. The results and subject experience were evaluated.

TABLE 1

| | | Number | | Number of | | Number | |
|---|---|---|---|---|---|---|---|
| | | of | | Subjects with | | of | |
| | Number | Subjects | | Skin Stinging, | | Subjects | Odor |
| | of | Showing | Effective | Redness, | Discomfort | Accepting | Acceptance |
| Composition | Subjects | Effects | Rate | Swelling, etc. | Rate | Odor | Rate |
| Comparative | 20 | 14 | 70% | 4 | 20% | 11 | 55% |
| Example 4 | | | | | | | |
| Example 8 | 20 | 17 | 85% | 0 | 0 | 20 | 100% |
| Example 9 | 20 | 18 | 90% | 0 | 0 | 20 | 100% |
| Example 11 | 20 | 16 | 80% | 0 | 0 | 20 | 100% |

As shown in Table 1, the recombinant collagen ionic liquid compositions of Examples 8, 9, and 11 provided by the present disclosure had a significant effect on promoting hair growth, with an effective rate of 80% or higher. Compared to Comparative Example 4, Examples 8, 9, and 11 had superior safety and subject experience, with a 100% odor acceptance rate among the subjects, and no adverse reactions occurred during the use period.

### Test Example 3

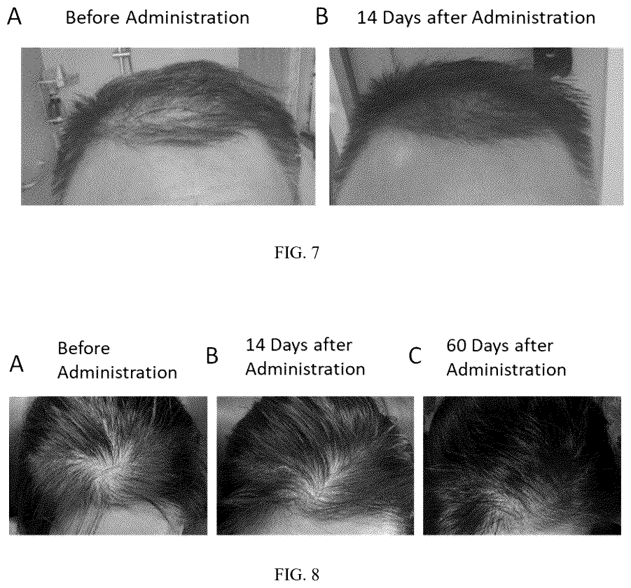

A 45-year-old male used the recombinant collagen ionic liquid composition of Example 8 seven times a week by applying it to the scalp. The composition was completely absorbed in 15 min without pressing. After continuous use for 14 days, short hair grew, and hair density increased (FIG. 7).

### Test Example 4

A 50-year-old female used the recombinant collagen ionic liquid composition of Example 8 seven times a week by applying it to the scalp. The composition was completely absorbed in 15 min without pressing. After continuous use for 60 days, the hair loss was significantly improved, and the hair density significantly increased (FIG. 8).

The results above indicate that the recombinant collagen ionic liquid composition provided by the present disclosure has good solubility, high transdermal efficiency, and low irritation. Furthermore, the method for producing the ionic liquid composition features a short preparation cycle and a simple process, and allows for continuous production. Therefore, the ionic liquid composition and its production method are of great significance.

Finally, it should be noted that the above examples are only used to illustrate the technical solutions of the present disclosure, but not to limit the present disclosure. Although the present disclosure is described in detail in conjunction with the foregoing examples, a person of ordinary skill in the art should understand that they can still modify the technical solutions described in the foregoing examples, or make equivalent substitutions for some or all of the technical features therein. These modifications or substitutions do not cause the essence of the corresponding technical solutions to depart from the scope of the technical solutions of the examples of the present disclosure.

1. A recombinant collagen ionic liquid composition, comprising, in parts by mass: 19.8 to 79.99 parts of a lysine citrate ionic liquid, 20 to 80 parts of glycerol, and 0.01 to 0.2 parts of a recombinant collagen; wherein the lysine citrate ionic liquid consists essentially of lysine and citric acid, in a molar ratio of the lysine to the citric acid of (4 to 1):(2 to 1), and the recombinant collagen includes a type XVII recombinant collagen.

2. The recombinant collagen ionic liquid composition according to claim 1, comprising, in parts by mass, 29.85 to 49.95 parts of the lysine citrate ionic liquid, 50 to 70 parts of glycerol, and 0.05 to 0.15 parts of the recombinant collagen.

3. The recombinant collagen ionic liquid composition according to claim 1, wherein the lysine citrate ionic liquid is produced by: mixing lysine, citric acid and water, subjecting the mixture to a reaction, and removing water after the reaction being completed to obtain the lysine citrate ionic liquid.

4. The recombinant collagen ionic liquid composition according to claim 3, wherein the reaction is performed at a temperature of 25° C. to 100° C. for 12 to 24 h.

5. The recombinant collagen ionic liquid composition according to claim 3, wherein the removing water is performed by distillation under reduced pressure.

6. The recombinant collagen ionic liquid composition according to claim 1, further comprising, in parts by mass, 0 to 30 parts of water.

7. A method for producing the recombinant collagen ionic liquid composition according to any one of claims 1 to 6, comprising: mixing all components in formulated amounts to obtain the recombinant collagen ionic liquid composition.

8. The method according to claim 7, wherein components other than the recombinant collagen are mixed first, and then the recombinant collagen is added and mixed.

9. Use of the recombinant collagen ionic liquid composition according to any one of claims 1 to 6 in the manufacture of a cosmetic for promoting hair follicle repair and hair growth.
